(19) United States
(12) Patent Application Publication
(10) Pub. No.: US 2020/0396566 A1
(43) Pub. Date: Dec. 17, 2020

(54) INTERACTIVE DANCE CONTEST

(71) Applicant: Discology, Inc., Irvine, CA (US)

(72) Inventors: Genesis Kim, San Francisco, CA (US); Jared Fishman, Palm Beach, FL (US)

(21) Appl. No.: 16/894,510

(22) Filed: Jun. 5, 2020

Related U.S. Application Data
(60) Provisional application No. 62/861,195, filed on Jun. 13, 2019.

(51) Int. Cl. H04W 4/029; A63B 24/00; A61B 5/11; G01S 5/00

(52) U.S. Cl. CPC H04W 4/029; A63B 24/0062; A63B 2225/50; G01S 5/0027; A61B 2562/0219; A61B 5/1118

(57) ABSTRACT

A motion-based dance system including a plurality of wireless devices, said wireless devices communicably coupled to a server. The wireless devices include a motion sensor and a location sensor. A server receives from the wireless devices position and motion information and compares them to predetermine values. Those values may indicate compliance with preset location and motion information such as a dance contest and the like. Performance information may be communicated back to the wireless devices thus allowing a wirelessly coupled dance contest or other movement activity to be performed remotely or under location and distance constraints. Certain embodiments may be coupled to an award system for recognizing and rewarding dancers for the amount of participation the dancer performs.

### PRIORITY

This application claims the benefit of co-pending provisional patent application 62/861,195 filed Jun. 13. 2019 by the same inventors which is included by reference as if fully set forth herein.

### BACKGROUND

The wireless world offers a great deal of opportunity to advance technology in many areas. Conventionally, with the threat of COVID-19, Virtual connection has become the norm, social isolation is at an all-time high with online user interactions. Presented herein as systems and methods for an intentional ritual that improves one's physical, social, and emotional well-being - while remaining socially distanced.

### SUMMARY

Disclosed herein is a motion-based dance system including a plurality of wireless devices, said wireless devices communicably coupled to a server. The wireless devices include a motion sensor and a location sensor. A server receives from the wireless devices position and motion information and compares them to predetermine values. Those values may indicate compliance with preset location and motion information such as a dance contest and the like. Performance information like location, times, duration, accelerometer movements, and the like, may be communicated back to the wireless devices thus allowing a wirelessly coupled dance contest or other movement activity to be performed remotely or under location and distance constraints. Certain embodiments may be coupled to an award system for recognizing and rewarding dancers for the amount of participation the dancer performs.

Some embodiments may include server instructions to operate contests wherein the remote user or dancer receives specific location information to enter the contest. This may include geographic location information which may operate to have dancers congregate or to disperse. Some embodiments may provide dancers instructions to maintain a distance from other dancers or participants.

The remote users may transmit motions and location information back to the server. The server may apply algorithms comparing the motion and location information received from the remote users to effectuate a scoring scheme. For example, and without limitation, dancers with the most moves may be rewarded or publicly recognized. Some embodiments may include a public display which allows for onlookers to see how the dancers are performing in relation to each other.

### BRIEF DESCRIPTION OF THE DRAWINGS

FIG. 1 shows a functional block diagram of a client server system that may be employed for some embodiments according to the current disclosure.

FIG. 2 illustrates a functional diagram of an embodiment of a dance move system according to the current disclosure.

FIG. 3 is a flow chart containing steps that may be employed in certain embodiments.

### DESCRIPTION

### Generality of Invention

This application should be read in the most general possible form. This includes, without limitation, the following:

References to specific techniques include alternative and more general techniques, especially when discussing aspects of the invention, or how the invention might be made or used.

References to “preferred” techniques generally mean that the inventor contemplates using those techniques, and thinks they are best for the intended application. This does not exclude other techniques for the invention, and does not mean that those techniques are necessarily essential or would be preferred in all circumstances.

References to contemplated causes and effects for some implementations do not preclude other causes or effects that might occur in other implementations.

References to reasons for using particular techniques do not preclude other reasons or techniques, even if completely contrary, where circumstances would indicate that the stated reasons or techniques are not as applicable.

Furthermore, the invention is in no way limited to the specifics of any particular embodiments and examples disclosed herein. Many other variations are possible which remain within the content, scope and spirit of the invention, and these variations would become clear to those skilled in the art after perusal of this application.

### Lexicography

The terms “effect”, “with the effect of” (and similar terms and phrases) generally indicate any consequence, whether assured, probable, or merely possible, of a stated arrangement, cause, method, or technique, without any implication that an effect or a connection between cause and effect are intentional or purposive.

The term “relatively” (and similar terms and phrases) generally indicates any relationship in which a comparison is possible, including without limitation “relatively less”, “relatively more”, and the like. In the context of the invention, where a measure or value is indicated to have a relationship “relatively”, that relationship need not be precise, need not be well-defined, need not be by comparison with any particular or specific other measure or value. For example and without limitation, in cases in which a measure or value is “relatively increased” or “relatively more”, that comparison need not be with respect to any known measure or value, but might be with respect to a measure or value held by that measurement or value at another place or time.

The term “substantially” (and similar terms and phrases) generally indicates any case or circumstance in which a determination, measure, value, or otherwise, is equal, equivalent, nearly equal, nearly equivalent, or approximately, what the measure or value is recited. The terms “substantially all” and “substantially none” (and similar terms and phrases) generally indicate any case or circumstance in which all but a relatively minor amount or number (for “substantially all”) or none but a relatively minor amount or number (for “substantially none”) have the stated property. The terms “substantial effect” (and similar terms and phrases) generally indicate any case or circumstance in which an effect might be detected or determined.

The terms “this application”, “this description” (and similar terms and phrases) generally indicate any material shown or suggested by any portions of this application, individually or collectively, and include all reasonable conclusions that might be drawn by those skilled in the art when this application is reviewed, even if those conclusions would not have been apparent at the time this application is originally filed.

The term “virtual machine” or “VM” generally refers to a self-contained operating environment that behaves as if it is a separate computer even though is is part of a separate computer or may be virtualized using resources form multiple computers.

### DETAILED DESCRIPTION

Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

### System Elements

### Processing System

The methods and techniques described herein may be performed on a processor-based device. The processor-based device will generally comprise a processor attached to one or more memory devices or other tools for persisting data. These memory devices will be operable to provide machine-readable instructions to the processors and to store data. Certain embodiments may include data acquired from remote servers. The processor may also be coupled to various input/output (I/O) devices for receiving input from a user or another system and for providing an output to a user or another system. These I/O devices may include human interaction devices such as keyboards, touch screens, displays and terminals as well as remote connected computer systems, modems, radio transmitters and handheld personal communication devices such as cellular phones, “smart phones”, digital assistants and the like.

The processing system may also include mass storage devices such as disk drives and flash memory modules as well as connections through I/O devices to servers or remote processors containing additional storage devices and peripherals.

Certain embodiments may employ multiple servers and data storage devices thus allowing for operation in a cloud or for operations drawing from multiple data sources. The inventor contemplates that the methods disclosed herein will also operate over a network such as the Internet, and may be effectuated using combinations of several processing devices, memories and I/O. Moreover, any device or system that operates to effectuate techniques according to the current disclosure may be considered a server for the purposes of this disclosure if the device or system operates to communicate all or a portion of the operations to another device.

The processing system may be a wireless device such as a smart phone, personal digital assistant (PDA), laptop, notebook and tablet computing devices operating through wireless networks. These wireless devices may include a processor, memory coupled to the processor, displays, keypads, Wi-Fi, Bluetooth, GPS and other I/O functionality. Alternatively, the entire processing system may be self-contained on a single device in certain embodiments.

The methods and techniques described herein may be performed on a processor-based device. The processor-based device will generally comprise a processor attached to one or more memory devices or other tools for persisting data. These memory devices will be operable to provide machine-readable instructions to the processors and to store data, including data acquired from remote servers. The processor will also be coupled to various input/ output (I/O) devices for receiving input from a user or another system and for providing an output to a user or another system. These I/O devices include human interaction devices such as keyboards, touchscreens, displays, pocket pagers and terminals as well as remote connected computer systems, modems, radio transmitters and handheld personal communication devices such as cellular phones, “smart phones” and digital assistants.

The processing system may also include mass storage devices such as disk drives and flash memory modules as well as connections through I/O devices to servers containing additional storage devices and peripherals. Certain embodiments may employ multiple servers and data storage devices thus allowing for operation in a cloud or for operations drawing from multiple data sources. The inventor contemplates that the methods disclosed herein will operate over a network such as the Internet, and may be effectuated using combinations of several processing devices, memories and I/ O.

The processing system may be a wireless device such as a smart phone, personal digital assistant (PDA), laptop, notebook and tablet computing devices operating through wireless networks. These wireless devices may include a processor, memory coupled to the processor, displays, keypads, Wi-Fi, Bluetooth, GPS and other I/O functionality. The wireless devices such as smartphones, headsets and other wearable devices may include motion detection circuits such as accelerometers, inertia switch detectors, audio sensing and magnetometers.

### Client Server Processing

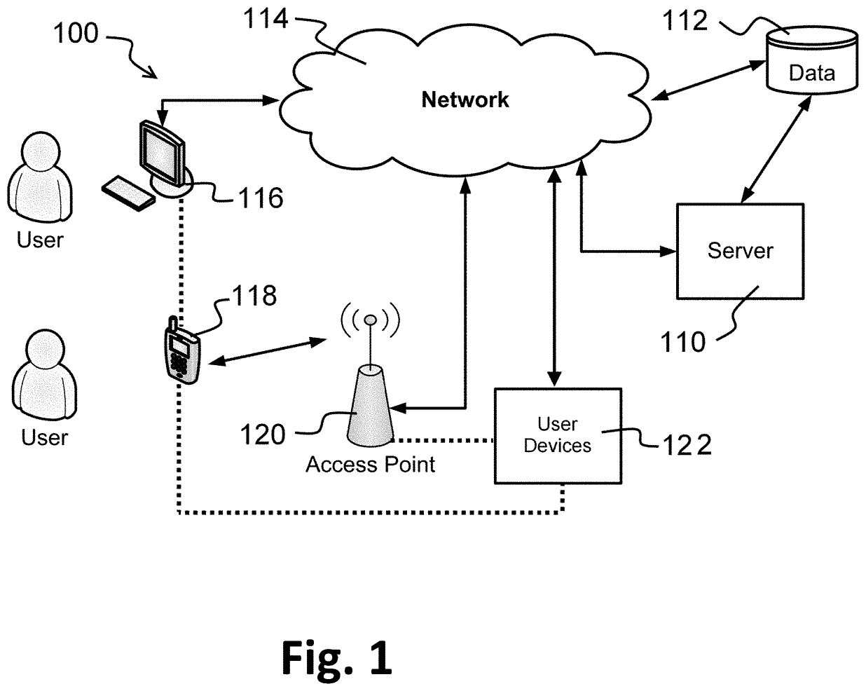

FIG. 1 shows a functional block diagram of a client server system 100 that may be employed for some embodiments according to the current disclosure. In the FIG. 1 a server 110 is coupled to one or more databases 112 and to a network 114. The network may include routers, hubs and other equipment to effectuate communications between all associated devices. A user accesses the server by a computer 116 communicably coupled to the network 114. The computer 116 includes a sound capture device such as a microphone (not shown). Alternatively, the user may access the server 110 through the network 114 by using a smart device such as a telephone or PDA 118. The smart device 118 may connect to the server 110 through an access point 120 coupled to the network 114. The mobile device 118 includes a sound capture device such as a microphone.

A system as disclosed in FIG. 1 may include one or more user devices 122 coupled to the network 114 directly, through the access point 120, or directly to remote processing devices. For example, and without limitation, a virtual reality (VR), or game controller may be coupled to a processing device for getting user input. This coupling may be wireless using technologies such as Bluetooth.

Conventionally, client server processing operates by dividing the processing between two devices such as a server and a smart device such as a cell phone or other computing device. The workload is divided between the servers and the clients according to a predetermined specification. For example, in a “light client” application, the server does most of the data processing and the client does a minimal amount of processing, often merely displaying the result of processing performed on a server.

According to the current disclosure, client-server applications are structured so that the server provides machine-readable instructions to the client device and the client device executes those instructions. The interaction between the server and client indicates which instructions are transmitted and executed. In addition, the client may, at times, provide for machine readable instructions to the server, which in turn executes them. Several forms of machine-readable instructions are conventionally known including applets and are written in a variety of languages including Java and JavaScript.

Client-server applications also provide for software as a service (SaaS) applications where the server provides software to the client on an as needed basis.

In addition to the transmission of instructions, client-server applications also include transmission of data between the client and server. Often this entails data stored on the client to be transmitted to the server for processing. The resulting data is then transmitted back to the client for display or further processing.

One having skill in the art will recognize that client devices may be communicably coupled to a variety of other devices and systems such that the client receives data directly and operates on that data before transmitting it to other devices or servers. Thus, data to the client device may come from input data from a user, from a memory on the device, from an external memory device coupled to the device, from a radio receiver coupled to the device or from a transducer coupled to the device. The radio may be part of a wireless communications system such as a “Wi-Fi” or Bluetooth receiver. Transducers may be any of a number of devices or instruments such as thermometers, pedometers, health measuring devices and the like.

A client-server system may rely on “engines” which include processor-readable instructions (or code) to effectuate different elements of a design. Each engine may be responsible for differing operations and may reside in whole or in part on a client, server or other device. As disclosed herein a display engine, a data engine, an execution engine, a user interface (UI) engine and the like may be employed. These engines may seek and gather information about events from remote data sources.

This methods and techniques in this disclosure may be effectuated using conventional programming tools including database tools for collecting, storing and searching through structured data. Moreover, web-based programming techniques may be employed to collect information, display results and allocate compensation. Accordingly, software engines may be created to effectuate these methods and techniques, either in whole or part, depending on the desired embodiment.

Commercially available modules such as the Raspberry PI include accelerometers, and wireless communications as well as on-board programmability. In some embodiments, these devices may be programmed and distributed to dancers who wear them on their clothing such as in an armband or belt clip fashion. These may be programmed to detect a dance move and transmit that information to a local wireless network such as Wi-fi. Similarly, a smart phone with a motion detector may be programmed to effectuate a similar result - programmatically detecting and recording movements such as dance moves.

References in the specification to “one embodiment”, “an embodiment”, “an example embodiment”, etc., indicate that the embodiment described may include a particular feature, structure or characteristic, but every embodiment may not necessarily include the particular feature, structure or characteristic. Moreover, such phrases are not necessarily referring to the same embodiment. Further, when a particular feature, structure or characteristic is described in connection with an embodiment, it is submitted that it is within the knowledge of one of ordinary skill in the art to effect such feature, structure or characteristic in connection with other embodiments whether or not explicitly described. Parts of the description are presented using terminology commonly employed by those of ordinary skill in the art to convey the substance of their work to others of ordinary skill in the art.

### Data Capture

Recording of motion requires some form of data capture methodology. As disclosed herein a wearable device or other device coupled to a dancer will be motion-sense enable. Included on the device will be circuitry to operate the motion sensing and provide the information wirelessly to a remote server. This may be through continuous polling from a master node, by collection and later uploading the data, or similar means to meet the objectives of the embodiment.

### Collector Calibration

Before, during, or after the collection process, a calibration procedure may be invoked. This procedure may provide for a determination on when a move is an actual dance move or when it is merely some other, more routine, motion. In some embodiments a pre-determined default amount of movement may be set such that a dance must move more than the default amount to be recorded as a dance move.

### Environment

Information about the local environment may be gathered to indicate a dancer is in a pre-determined area. This information may be determined using GPS or other position sensing or locating technique to ensure movement outside of the specified area is not counted as a dance move. Moreover, the distances between participants such as dancers, staff, bystanders, and the like, may be determined using GPS to ensure dancers are in a predefined area and not too close to another dancer.

### System Overview

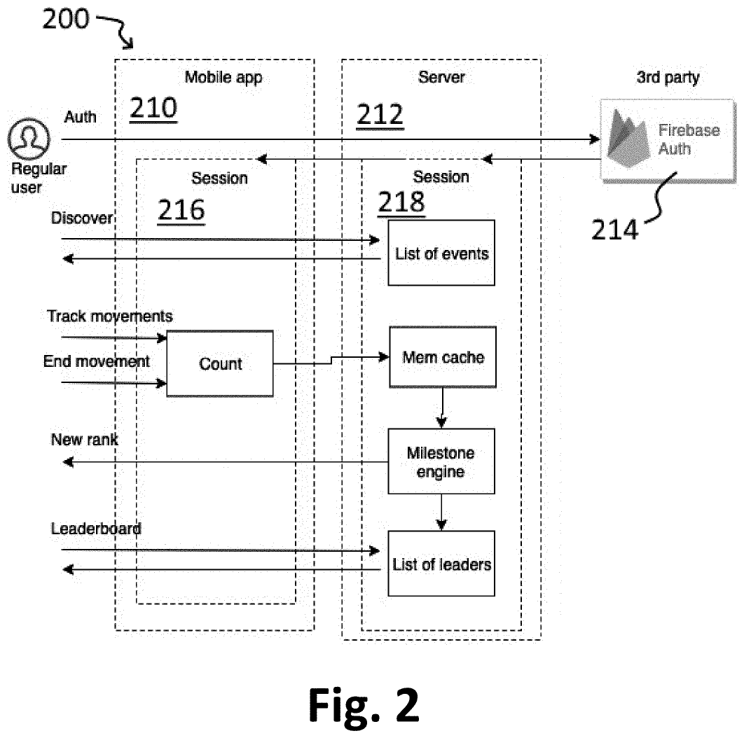

FIG. 2 illustrates a functional diagram of an embodiment of a dance move system 200 according to the current disclosure. The system elements include a mobile app 210, a server 212 and a third part authentication system 214 such as the commercially available firebase authentication.

The server 212 will host data for multiple sessions 218 and expose that information to a related session 216 residing on the mobile app 210. The session information may include a list of events, a memory cache, a milestone engine and a list of leaders.

In operation the events may be promotional events wherein a group of users dance as part of a contest. Contest rules and parameters may be transmitted to participants in advance. The dance moves for each user are counted and the dancer with the most moves is the winner of the contest.

The memory cache operates to record the move information provided by a counter in the mobile app 216. A processor (not shown) evaluates the dance moves and records any milestones to determine ranking. These milestones may be for a single contest or across multiple contests for a single user or identified group of users. The milestone engine further provides a list of leaders which may be a public display.

An application programming interface (API) may expose to the mobile app 210 and other users the elements described herein. For example, and without limitation, the API may allow third parties to query the milestone engine or list of leaders to display the results on a web page or, in the event of a public event, to show the list of leaders on a leaderboard exposed to all the dancers. For sponsored events, this may incentivize dancer to try harder to win the contest.

The API may also expose the information for use in artistic and graphical displays. For example, and without limitation, visuals behind a DJ, such as white lights on a screen, may become reactive to dance moves collected by event participants. 100,000 dance moves a second might cause a bright, flashing light as compared to 100 dance moves a second.

The mobile app may include processor readable instructions directing the processor to interact with a motion sensor to determine dance moves. The mobile app 210 operates to allow a dancer to discover and join a session, report the results of the movements to the server 212, display standings and rank to a mobile app 210 user, and, in some embodiments, couple to a third-party software for reporting information.

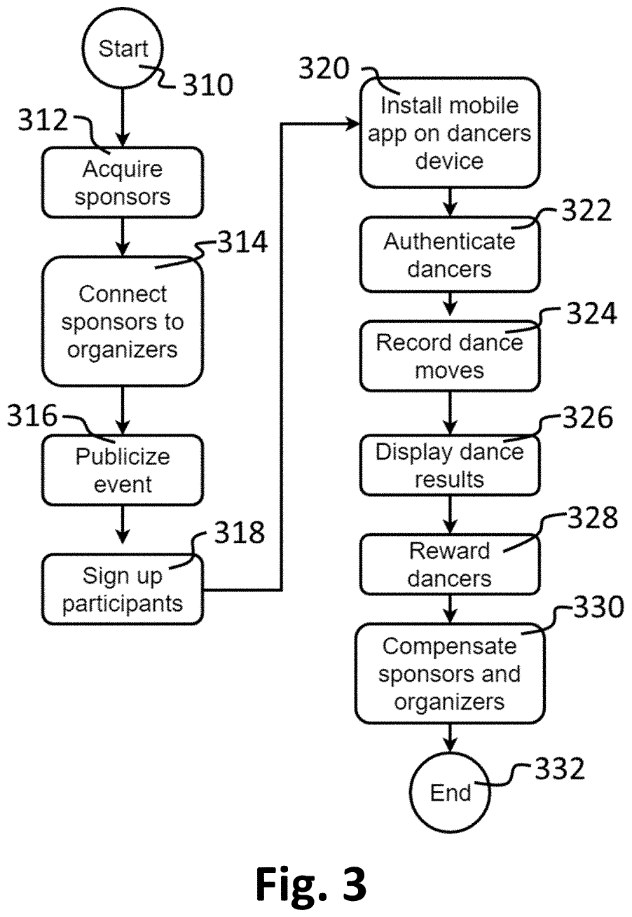

FIG. 3 is a flow chart containing steps that may be employed in certain embodiments. In FIG. 3 the method starts at a flow label 310.

At a step 312 sponsors are acquired. The sponsors are the people or entity that want to be associated with the fundraising, usually by supplying money or other awards to winners in exchange for recognition.

At a step 314 the sponsors are connected to the organizers. Organizers are the people or entity that hosts and operates the event.

At a step 316 the event is publicized.

At a step 318 participants are signed up or entered into the contest.

At a step 320 a mobile app is installed on dancer's device.

At a step 322 dancers are authenticated by verifying their attendance in the contest at an acceptable location.

At a step 324 dances moves are detected and recorded.

At a step 326 dance moves are displayed. This may include displaying them to other contestants either on mobile devices or on a public display. Scoring may be the volume or number of moves or the quantity of motion.

At a step 328 dancers are rewarded. There may be multiple categories for rewards such as, but not limited to, most moves, most moved in a time period, longest time dancing, etc. . . .

At a step 330 sponsors and organizers apply appropriate compensation, which may include sponsors paying organizers and any associated charity.

At a step 332 the method ends.

In some embodiments artists and songs may be added to an event. Collectively the artists, events, sponsors, organizers and users may be referred to as a campaign. The objective of the campaign is to raise money for a purpose. In these embodiments dance moves for a particular artist, song or collection of songs may be included in a campaign and included in the resulting dataset.

In some embodiments dancers may be authenticated to a predetermined location using GPS. The location may be a defined dancing are including public locations such as parks. The location may be parsed into smaller regions which allow only a single dancer in a defined area. Other embodiments may allow for closing related people, such as those living together, to share a predefined space for dancing.

In some embodiment advertisers with appropriate indicia for promoting products or services may be include as part of an event or campaign. In these embodiments, advertising may be tied to specific dance counts. For example, and without limitation, if a dance count on a per event, per song or per artists is achieved in the event, the promotional indicia may be displayed or broadcast to the event participants.

What is claimed:

1. A method for a remote contest including:
transmitting to a first wireless device contest information said contest information including at least one of a start time, a first location information, or a rule;
receiving, at a server, dancer information from the first wireless device, said dancer information including either a second location information or a motion information;
comparing the dancer information to the contest information and responding to the first wireless device in response to said comparing;
receiving from a second wireless device a second motion information;
comparing the first motion information to the second motion information, and
transmitting to the first and second wireless devices the results of said comparing.

2. The method of claim 1 further including:
electronically displaying the dancer information.

3. The method of claim 1 wherein the second location information is sensed by a global positioning system sensor.

4. The method of claim 1 wherein the first wireless device is a mobile phone.

5. The method of claim 1 further including:
transmitting to either the first or second wireless device award information.

5. The method of claim 1 further including:
transmitting to either the first or second wireless device sponsor information.

6. The method of claim 1 further including:
comparing the first motion information to a known value, and scoring counting motion in response to the comparing

7. A motion-based dance system including:
a plurality of wireless devices, said wireless devices communicably coupled to a server;
said wireless devices including a motion sensor operable to sense movement of the wireless devices;
said wireless devices further including a location sensor;
said server including non-transitory processor-readable instructions directing the server to perform a method including:
receiving from the wireless devices position and motion information;
comparing said position and motion information to predetermine values, and
communicating to the wireless devices information in response to said comparing.

8. The system of claim 7 wherein the comparing said position and motion information to predetermine values includes ranking motion information by volume.

9. The system of claim 7 wherein the method further includes:
calibrating the motion information against pre-determined default amount of movement.

10. The system of claim 7 wherein said communicating to the wireless devices includes contest rank information.

11. The system of claim 7 wherein said communicating to the wireless devices includes contest sponsorship information.

12. A processor-readable memory device including non-transitory processor-readable instruction directing a processor to perform a method including:
transmitting to a first wireless device contest information said contest information including at least one of a start time, a first location information, or a rule;
receiving, at a server, dancer information from the first wireless device, said dancer information including either a second location information or a motion information;
comparing the dancer information to the contest information and responding to the first wireless device in response to said comparing;
receiving from a second wireless device a second motion information;
comparing the first motion information to the second motion information, and
transmitting to the first and second wireless devices the results of said comparing.

13. The device of claim 12 wherein the method further includes electronically displaying the dancer information.

14. The device of claim 12 wherein the second location information is sensed by a global positioning system sensor.

15. The device of claim 12 wherein the first wireless device is a mobile phone.

16. The device of claim 12 wherein the method further includes:
transmitting to either the first or second wireless device award information.

17. The device of claim 12 wherein the method further includes:
transmitting to either the first or second wireless device sponsor information.
